(19) United States
(12) Patent Application Publication
(10) Pub. No.: US 2022/0040123 A1
(43) Pub. Date: Feb. 10, 2022

(54) ANTI-HELICOBACTER FOOD COMPOSITION COMPRISING BETA-CARYOPHYLLENE

(71) Applicant: Sung Kyu KIM, Chungcheongnam-do (KR)

(72) Inventors: Sung Kyu KIM, Chungcheongnam-do (KR); So Young PARK, Chungcheongnam-do (KR); Sa Hyun KIM, Seoul (KR); Hyun Jun WOO, Daegu (KR)

(21) Appl. No.: 17/419,101

(22) PCT Filed: Dec. 11, 2019
(86) PCT No.: PCT/KR2019/017446

§ 371 Date: Jun. 28, 2021

(30) Foreign Application Priority Data
Dec. 28, 2018 (KR) 10-2018-0172268

(51) Int. Cl. A61K 31/015; A23L 33/10; A23C 9/13; A61P 31/04

(52) U.S. Cl. CPC A61K 31/015; A61P 31/04; A23C 9/13; A23L 33/10

(57) ABSTRACT

An anti-Helicobacter food composition having excellent ability to eradicate Helicobacter pylori present in the stomach by using beta-caryophyllene is provided. The anti-Helicobacter food composition contains beta-caryophyllene having a purity of 90% or higher, obtained by distilling clove oil at 250 to 270° C., in an amount corresponding to a daily adult intake of 100 to 2,500 mg.

### BACKGROUND

The present invention relates to an anti-Helicobacter food composition having excellent ability to eradicate Helicobacter pylori present in the stomach by using beta-caryophyllene.

Helicobacter pylori was successfully isolated and cultured by Australian researchers, Dr. Marshall and Dr. Warren, in 1983, and was named by them. Many papers have revealed that Helicobacter pylori is detected in the hygienic examinations of patients with gastritis, gastric ulcer, and duodenal ulcer at high rate, so this bacterium has been recognized as a risk factor for gastric ulcer, gastritis, gastric cancer, and duodenal ulcer.

Helicobacter pylori is a gram-negative bacillus which colonizes the junctions between the epithelial cells of the gastric mucosa. It can grow under microaerobic conditions with optimal pH 7.0 to 7.4 and temperature 30 to 37° C. The virulence factors for Helicobacter pylori are CagA secreted for survival in the strong acidic conditions of the gastric juice produced from the stomach, SecA protein for secretion of VacA, flagella for maintaining motility, and outer membrane proteins to facilitate adhesion to the epithelial cells of the gastric mucosa. Another virulence factor, urease metabolizes urea in the gastric musical tissue into ammonia and carbon dioxide to alkalize the cell strain and neutralize the strong acid selected from the stomach, creating conditions acceptable for survival of H. pylori.

The representative treatment method for Helicobacter pylori relies on antibiotics such as metronidazole and amoxicillin. It has been reported that the repeated use of these antibiotics can cause increased antibiotic resistance and various side effects. Sustained efforts have been made to explore extracts and active ingredients for suppressing Helicobacter pylori using a variety of natural materials. It has been confirmed that different polyphenols extracted from grapes and apple peels or isolated from blackberry leaves can inhibit the growth of Helicobacter pylori. A variety of flavonoid compounds from thyme or the like have also been reported to have excellent inhibitory activity.

In addition, the recent patents and reports related to Helicobacter pylori include a method for preparing a fortified pure extract having an inhibitory activity against H. pylori, and a product containing the pure extract as an active ingredient (Korean Patent Application Publication No. 10-2012-0053352); an anti-Helicobacter composition containing a green algae extract (Korean Patent Application Publication No. 10-2011-0023844); a pharmaceutical composition and a health food for preventing or treating Helicobacter pylori infectious disease that contain a cinnamon extract as an active ingredient (Korean Patent Application Publication No. 10-2011-0117491); and a composition for preventing or treating gastrointestinal diseases that contains a Chrysanthemum zawadskii extract or fraction as an active ingredient (Korean Patent Application Publication No. 10-2010-0044433).

### SUMMARY OF THE INVENTION

It is an object of the present invention to provide an anti-Helicobacter food composition having excellent ability to eradicate Helicobacter pylori present in the stomach by using beta-caryophyllene.

An anti-Helicobacter food composition according to the present invention contains beta-caryophyllene having a purity of 90% or higher, obtained by distilling clove oil at 250 to 270° C., in an amount corresponding to a daily adult intake of 100 to 2,500 mg.

The anti-Helicobacter food composition according to the present invention has excellent bactericidal activity against Helicobacter pylori present in the stomach.

### BRIEF DESCRIPTION OF DRAWINGS

FIG. 1 is a schematic diagram showing a process of preparing animals infected with H. pylori.

FIG. 2 is a graph showing the minimum concentration of beta-caryophyllene for the inhibition of growth of H. pylori.

FIG. 3 depicts a photograph showing the inhibition of expression of H. pylori-specific gene according to the present invention.

FIG. 4 depicts gastric mucosa and submucosal layer photographs taken depending on the concentration of beta-caryophyllene in order to confirm the H. pylori eradiation effect according to the present invention.

FIG. 5 is a graph showing the results of measuring the amounts of CagA, VacA and SecA proteins depending on the concentration of beta-caryophyllene in order to evaluate the inhibitory effect against H. pylori-secreted toxins (CagA, VacA, and SecA) according to the present invention.

FIG. 6 is a graph showing the changes in expression of cagA, vacA and secA genes depending on the concentration of beta-caryophyllene according to the present invention.

FIG. 7 is a graph showing the changes in expression of urease protein depending on the concentration of beta-caryophyllene according to the present invention.

FIG. 8 is a graph showing the changes in expression of alpA, alpB and babA genes depending on the concentration of beta-caryophyllene according to the present invention.

FIG. 9 is a graph showing the changes in expression of flhA and flgE genes depending on the concentration of beta-caryophyllene according to the present invention.

FIG. 10 is a graph showing the changes in expression of dnaA, dnaN, holB and polA genes depending on the concentration of beta-caryophyllene according to the present invention.

FIG. 11 is a graph showing the changes in expression of virB2, virB4, virB5, virB6, virB7, virB8, virB9, virB10 and virD4 genes depending on the concentration of beta-caryophyllene according to the present invention.

### DETAILED DESCRIPTION OF THE INVENTION

Hereinafter, embodiments of the present invention will be described with reference to the accompanying drawings.

The beta-caryophyllene of the present invention is preferably obtained by distilling clove oil (clove leaf oil or clove stem oil) at 250 to 270° C. and has a purity of 90% or higher. The beta-caryophyllene with a purity of less than 90% may be difficult to use as an anti-Helicobacter agent.

The food composition of the present invention preferably contains beta-caryophyllene in an amount corresponding to a daily adult intake of 100 mg or greater, preferably 100 to 2,500 mg.

The beta-caryophyllene-containing food composition of the present invention may be added to yogurt or lactic acid bacteria-fermented milk and formulated into a lactic acid bacteria-fermented beverage. In this regard, the beta-caryophyllene is preferably contained in an amount of 0.01 to 5 wt % based on the total weight of the yogurt or lactic acid bacteria-fermented milk.

This anti-Helicobacter food composition of the present invention is a composition containing beta-caryophyllene, and it can be prepared in a variety of ways so as to provide sustained, immediate, or delayed release. The beta-caryophyllene of the present invention may be included in the food composition by mixing or dilution with a carrier or encapsulation into capsules. Examples of carriers, typically available in formulation, include, but are not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil.

The food composition of the present invention may further contain ingredients that do not impair the bactericidal activity and growth inhibitory activity of beta-caryophyllene against H. pylori in the stomach. These ingredients include, for example, lubricants, wetting agents, sweeteners, flavoring agents, emulsifiers, suspending agents, and preservatives.

The food composition of the present invention may be prepared in the form of a unit dose or packaged in a multi-dose container by formulation using carriers and/or excipients according to a method that may be easily carried out by those skilled in the art. In this case, the formulation may be in the form of solution, suspension, syrup or emulsion in oil or aqueous medium; or in the form of extract, powder, granule, tablet, or capsule. It may further contain a dispersing agent or a stabilizing agent.

Hereinafter, a detailed description will be given as to the examples of the present invention. It should be apparent to those skilled in the art that these examples are given only for the purpose of more specific illustration of the present invention and are not construed to limit the scope of the present invention according to the subject matter of the present invention.

### Experimental Example 1: Culture of H. pylori

For H. pylori culture, an H. pylori (ATCC 49503) strain was inoculated on a Brucella agar medium containing 10% bovine serum and antibiotics (vancomycin, esfsuldin, trimethoprim, and amphotericin B), and cultured in an anaerobic incubator which maintains 10% CO₂, 5% O₂, 85% N₂, and 97% or more humidity, at 37° C. for 72 hours.

### Experimental Example 2: Preparation of Animals Infected with H. pylori

Mongolian gerbils were infected with a Helicobacter pylori strain (ATCC 49503) and then subjected to two weeks of acclimatization. To this end, the animals were fasted for 12 hours before and after inoculation with the Helicobacter pylori strain. 500 μl (1×10⁹ CFU) of a bacterial suspension was prepared by suspending H. pylori in sterile physiological saline to a concentration of 2×10⁹ CFU/ml and introduced directly into the stomach by using a feeding needle. For stable infection, the H. pylori strain was dispensed three times at 48 hour-intervals.

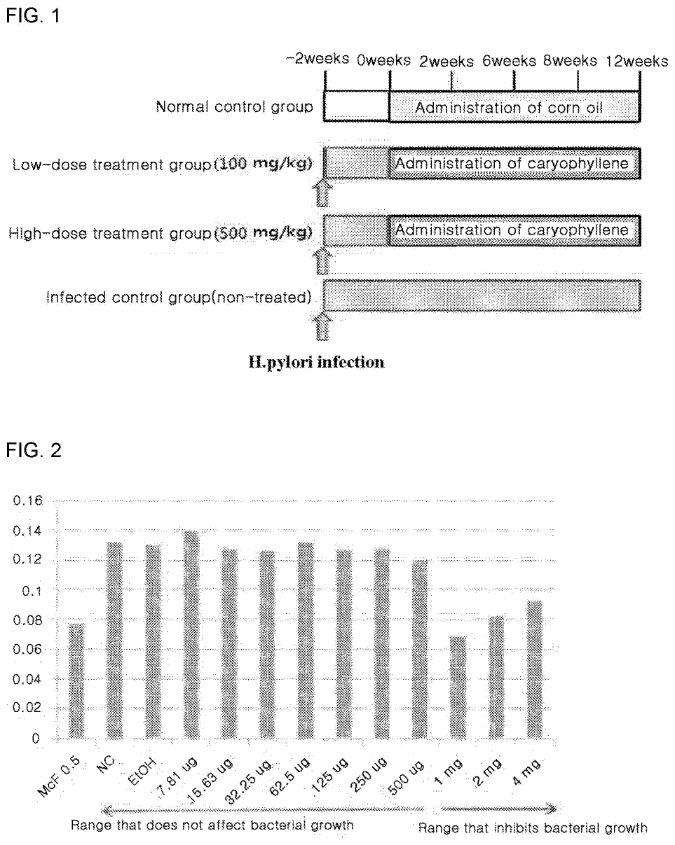

As shown in FIG. 1, the experimental animals were divided into four groups: non-infected group (NC) (not infected with H. pylori), infected group (HP) (infected with H. pylori), low-dose group (treated with 100 mg/kg of beta-caryophyllene), and high-dose group (treated with 500 mg/kg of beta-caryophyllene). Beta-caryophyllene was administered directly into the gastrointestinal at doses of 100 mg/kg and 500 mg/kg for the low and high dose groups, respectively, for 12 weeks after two weeks after infection with H. pylori. The same amount of corn oil as the beta-caryophyllene was administered to each of the non-infected group and the infected group.

(1) Determination of Minimum Concentration of Beta-Caryophyllene for Inhibition of Growth of H. pylori

In order to confirm the changes in expression of the pathogenic factors of Helicobacter pylori by administration of beta-caryophyllene, it is required to perform drug treatment at a concentration that does not inhibit the growth of Helicobacter pylori. This is because, when growth of the bacteria is inhibited, substances secreted by Helicobacter pylori also decrease, and hence the inhibitory effect of beta-caryophyllene against the pathogenic factors cannot be accurately evaluated.

For a minimum growth inhibitory concentration test, each of 7.81, 15.63, 31.25, 62.5, 125, 250 and 500 μg and 1, 2 and 4 mg of beta-caryophyllene was added to a medium obtained by adding 10% bovine serum to Mueller Hinton broth, and then cultured in a CO₂ incubator at a relative humidity of 100% for 72 hours. Each of the bacterial solutions cultured for 72 hours was measured for absorbance at 650 nm using a spectrophotometer to determine the minimum inhibitory concentration (MIC) of beta-caryophyllene, and the results are graphically shown in FIG. 2.

As shown in the graph of FIG. 2, it was confirmed that the minimum inhibitory concentration of beta-caryophyllene against H. pylori was 1 mg. The increase in absorbance at 2 mg and 4 mg was considered to be because of the effect of beta-caryophyllene that is an oil component. In the case of the control group (NC), H. pylori was cultured in the absence of beta-caryophyllene. Thus, the following experiment was performed at concentrations lower than the minimum inhibitory concentration, so that beta-caryophyllene did not affect the bacteria.

(2) Examination of Inhibition of Expression of H. pylori-Specific Genes

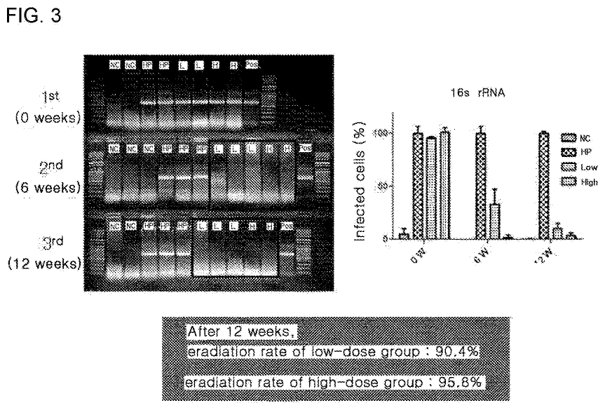

To confirm H. pylori infection, the stomach tissues of the four animal groups prepared in the H. pylori-infected animal preparation process shown in Experimental Example 2 were subjected to RT-PCR in order to examine whether H. pylori-specific 16S rRNA would be detected. The results are shown in FIG. 3.

As shown in the photograph of FIG. 3, As a result of examining Helicobacter pylori infection after sacrificing two animals for each group at week 0 of the experiment, it was confirmed that the animal model was successfully infected. As a result of observing Helicobacter pylori infection after sacrificing the experimental animals at intervals of 6 weeks, it was confirmed that the groups treated with 100 mg/kg and 500 mg/kg beta-caryophyllene showed Helicobacter pylori eradication rates of 90.4% and 95.8% at weeks 6 and 12.

(3) Evaluation of Effect on Helicobacter pylori Eradication in Gastric Mucosa and Submucosal Layer

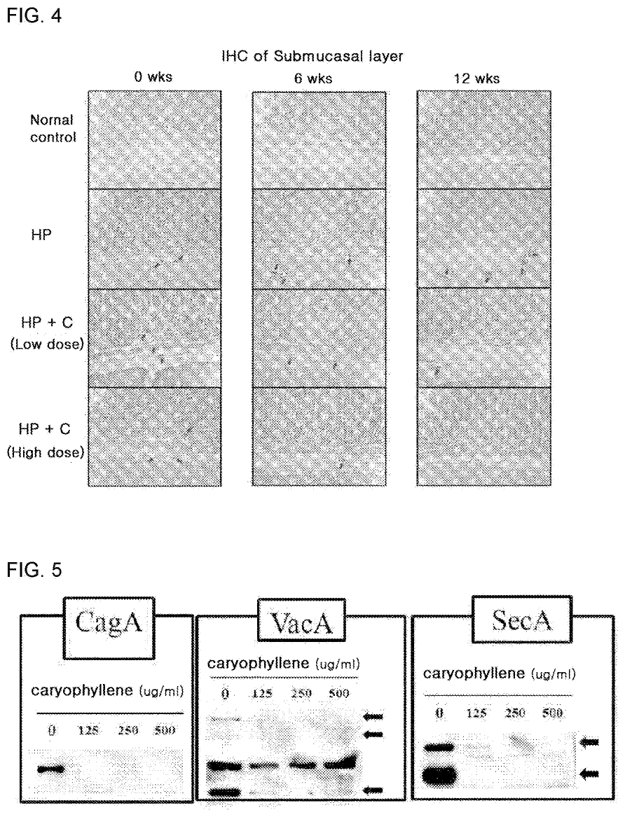

At weeks 0, 6 and 12 of the experiment, a paraffin block was prepared from with the stomach tissue of the infected animal model, and then stomach tissue slide sample with a thickness of 2 μm was prepared from the paraffin block using a microtome (Leica). For immunohistochemical staining, the slide was deparaffinized, heated with citrate buffer in a bath for 20 minutes, and then subjected to immunohistochemical staining using an IHC kit (Vectastain ABC kit, Vector). As primary antibody, Helicobacter pylori-specific antibody (anti-Helicobacter pylori rabbit IgG antibody [EPR10353], Abcam) was used. The stained slides were observed under a microscope at 200× and 400× magnifications to confirm the bacteriostatic effect against Helicobacter pylori in the gastric mucosa and submucosal layer, and the results are shown in FIG. 4.

As shown in the photograph of FIG. 4, as a result of detecting infected Helicobacter pylori in the gastric mucosa and submucosal layer tissue at week 0 of the experiment by immunohistochemical staining, it was confirmed that Helicobacter pylori was detected in all the groups (HP, low dose, and high dose) other than the non-infected group. In addition, it was confirmed that, at week 6 of the experiment, Helicobacter pylori infection was still detected in the infected group (HP), whereas Helicobacter pylori infection significantly decreased in the beta-caryophyllene-administered groups (low dose and high dose). In addition, as a result of immunohistochemical staining of the gastric mucosa at weeks 12 of the experiment, it was confirmed that Helicobacter pylori infection was still detected in the infected group (HP) to which beta-caryophyllene was not administered, whereas Helicobacter pylori was hardly detected in the beta-caryophyllene-administered groups (low dose and high dose). From these results, it was confirmed that, after infection, the group (HP) to which beta-caryophyllene was not administered had more severe infection, but in the beta-caryophyllene-administered groups (low dose and high dose), Helicobacter pylori that infected the submucosal layer was significantly removed.

Therefore, based on the results of the experiment on inhibition of the Helicobacter pylori-specific gene and the results of immunohistochemical staining of the stomach and submucosal layer using Helicobacter pylori-specific antibody, it was confirmed that beta-caryophyllene could effectively inhibit Helicobacter pylori infection in the experimental animals.

### Experimental Example 3: Experiment for Analysis of Expression of H. pylori Pathogenic Factors

(1) Evaluation of Inhibitory Effect Against Secretion of H. pylori Toxins (CagA, VacA and SecA)

To evaluate the effect of beta-caryophyllene treatment on the production of toxins secreted by Helicobacter pylori, Helicobacter pylori was treated with beta-caryophyllene, and then the amounts of CagA, VacA and SecA proteins were analyzed by Western blotting.

For Western blotting, samples obtained by treating Helicobacter pylori with beta-caryophyllene at concentrations of 0, 125, 250 and 500 μg/ml and culturing the treated Helicobacter pylori for 3 days were lysed with RIPA lysis buffer (Millipore) on ice for 30 minutes and then centrifuged. The supernatants were collected, and then proteins therein were quantified using a spectrophotometer (Infinite M200, TECAN).

Equal amounts of protein samples were loaded and electrophoresed on 10% SDS polyacrylamide gel, and the protein fractions were transferred to nitrocellulose membranes (PALL) and reacted with CagA (mouse monoclonal IgG antibody, Santa Cruz), VacA (rabbit polyclonal IgG antibody, Santa Cruz), SecA (rabbit polyclonal IgG antibody, produced by the applicant) and anti-Helicobacter pylori (rabbit polyclonal IgG antibody, produced by the applicant) antibodies at 4° C. for 16 hours. After completion of the primary antibody reaction, secondary blocking (1 hr/RT) was performed. As secondary antibodies, donkey anti-rabbit IgG-HRP, donkey anti-mouse IgG-HRP or donkey anti-goat IgG-HRP (Santa Cruz) was used depending on the primary antibody. The reaction products were treated by an enhanced chemiluminescence kit (Thermo), and then analyzed by Chemidoc (Fusion solo, Vilber Lourmat).

As a result of the Western blotting experiment, it could be confirmed that, when the H. pylori culture was various concentrations (0 to 500 μg/ml) of beta-caryophyllene (control, 125, 250, and 500 μg/ml), secretion of CagA, VacA and SecA was inhibited as shown in FIG. 5.

In addition, in order to examine changes in expression of cagA, vacA and secA genes, H. pylori was treated with beta-caryophyllene at concentrations of 31.25, 62.5, 125, 250 and 500 μg/ml, and then RT-PCR was performed.

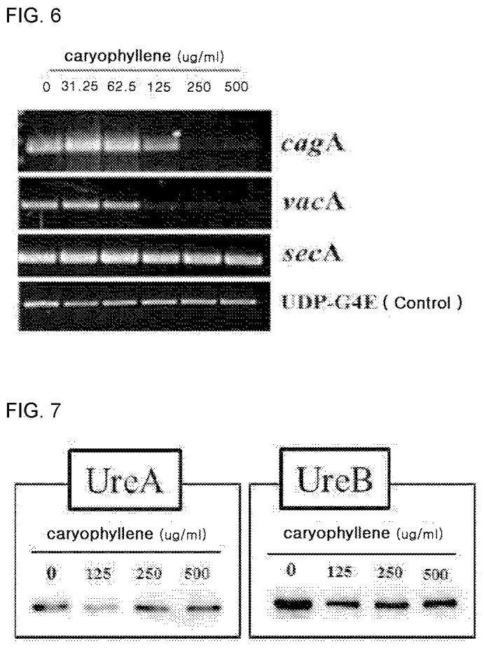

As a result of the RT-PCR experiment, as shown in FIG. 6, it could be observed that, when beta-caryophyllene was added to the H. pylori culture, transcription of the toxin protein genes cagA and vacA tended to be inhibited. The addition of beta-caryophyllene did not affect the transcription of other genes, including UDP-galactose 4-epimerase used as a control.

As shown in FIG. 6, it is concluded that beta-caryophyllene inhibits the secretion of toxin proteins by H. pylori, and also inhibits the expression of the toxin proteins.

(2) Evaluation of Inhibitory Effect Against Urease Production and Secretion of H. pylori

In order to evaluate the effect of beta-caryophyllene treatment on the inhibition of H. pylori urease, H. pylori was treated with beta-caryophyllene at concentrations of 125, 250 and 500 μg/ml, and changes in the expression of urease protein were analyzed, and the results are shown in FIG. 7.

As shown in FIG. 7, it was confirmed that the expression of urease A (Urease α, goat polyclonal IgG antibody, Santa Cruz) and urease B (Urease β, rabbit polyclonal IgG antibody, Santa Cruz) significantly decreased all the treatment concentrations of beta-caryophyllene, indicating that beta-caryophyllene has the effect of inhibiting the production and secretion of urease.

(3) Evaluation of Inhibitory Effect Against Adhesion Function of H. pylori

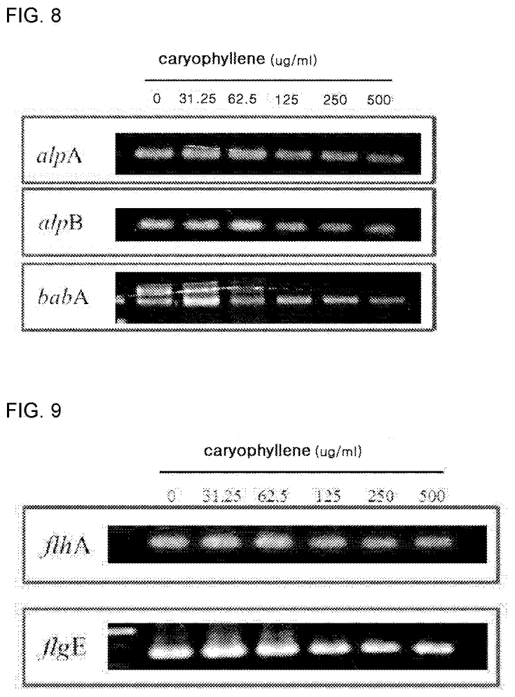

In order to evaluate the effect of beta-caryophyllene against the adhesion function of H. pylori, changes in the expression of the genes sabA, hopZ, hpaA, alpA, alpB and babA involved in the adhesion of H. pylori were analyzed, and the results are shown in FIG. 8. Specifically, H. pylori was treated with beta-caryophyllene at concentrations of 31.25, 62.5, 125, 250 and 500 μg/ml, and the expression of the genes was analyzed by RT-PCR.

As shown in FIG. 8, it was confirmed that the expression of the alpA, alpB and babA genes decreased. Thus, it could be confirmed that beta-caryophyllene has the effect of inhibiting the adhesion of H. pylori to gastric mucosa.

(4) Confirmation of Decreased Expression of flhA and flgE Genes Associated with Flagella of H. pylori

In order to evaluate the effect of beta-caryophyllene treatment on the inhibition of flagella of H. pylori, changes in the expression of the flhA, flaA, flaB and flgE genes which constitute the flagella or are associated with the function of the flagella were examined, and the results are shown in FIG. 9. Specifically, H. pylori was treated with beta-caryophyllene at concentrations of 31.25, 62.5, 125, 250 and 500 μg/ml, and expression of the genes was analyzed by RT-PCR.

As shown in FIG. 9, it was confirmed that expression of the flhA and flgE genes decreased.

(5) Evaluation of Inhibitory Effect of Beta-Caryophyllene Against Proliferation of H. pylori

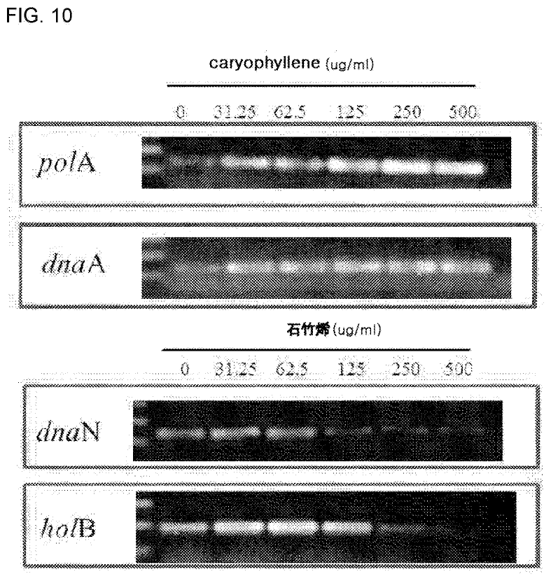

DNA replication is essential for the proliferation of Helicobacter pylori, and RNA synthesis (transcription process) is a process essential for protein synthesis in organisms. Thus, unless normal RNA synthesis occurs, normal life activity cannot be maintained. Accordingly, in order to evaluate the inhibitory effect of beta-caryophyllene against the proliferation of H. pylori, H. pylori was treated with beta-caryophyllene at concentrations of 31.25, 62.5, 125, 250 and 500 μg/ml, and changes in the expression of the DNA synthesis-related genes dnaA, dnaN, holB and polA were analyzed, and the results are shown in FIG. 10.

As shown in FIG. 10, it was confirmed that expression of the dnaA and polA increased and expression of the dnaN and holB genes significantly decreased. Thus, it was confirmed that beta-caryophyllene has the effect of suppressing Helicobacter pylori by affecting the genetic material-replication ability of Helicobacter pylori.

(6) Evaluation of Inhibitory Effect Against Toxin Injection Ability of Helicobacter pylori

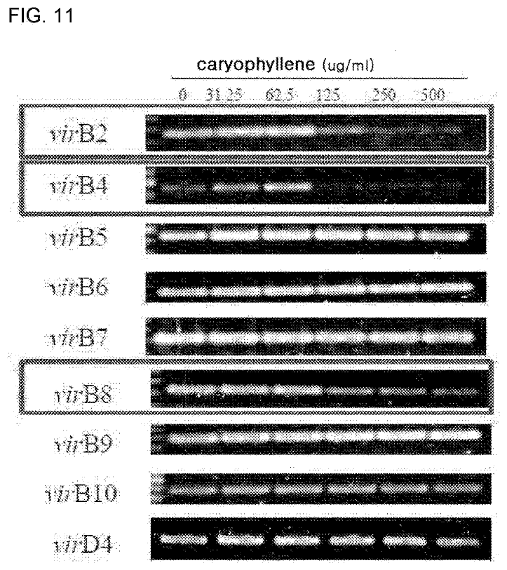

In order to evaluate the inhibitory effect of beta-caryophyllene against the CagA toxin injection ability of Helicobacter pylori, expression of virB2, virB4, virB5, virB6, virB7, virB8, virB9, virB10 and virD4 genes associated with the type IV secretion system (T4SS) structure required to inject the CagA toxin into a host cell was analyzed, and the results are shown in FIG. 11. Specifically, Helicobacter pylori was treated with beta-caryophyllene at concentrations of 31.25, 62.5, 125, 250 and 500 μg/ml, and expression of the genes was analyzed by RT-PCR.

As shown in FIG. 11, it was confirmed that expression of the virB2, virB4 and virB8 gene was significantly decreased by beta-caryophyllene treatment. Thus, it could be confirmed that beta-caryophyllene treatment has the effect of inhibiting the toxin injection ability of Helicobacter pylori.

### Experimental Example 4: Comparison of Eradiation Effect Between Beta-Caryophyllene and Antibiotics that are Used for Treatment of Helicobacter pylori Infection

In order to compare the Helicobacter pylori eradiation effect between beta-caryophyllene and the antibiotics that are used for treatment of Helicobacter pylori infection, an animal test was performed using the test groups and doses shown in Table 1 below.

Each test substance was suspended in an excipient according to the dose and administered to each mouse once a day at the same time every day for a total of 28 days in a dose of 5 ml per kg mouse (C57BL/6 mouse) by oral gavage method.

TABLE 1

| Group | Infection | Sample | Dose (ml/kg) |
|---|---|---|---|
| G1 | PBS | Corn oil | 5 |
| G2 | PBS | 0.5% CMC | 5 |
| G3 | H. pylori | Corn oil | 5 |
| G4 | H. pylori | 0.5% CMC | 5 |
| G5 | H. pylori | MTN + CLR + PPI | 5 |
| G6 | H. pylori | 100 mg/kg | 5 |
| G7 | H. pylori | 200 mg/kg | 5 |
| G8 | H. pylori | 500 mg/kg | 5 |

G1: Non-infection, corn oil,
G2: Non-infection,
G3: infection,
G4: infection,
G5: MTN (14.2 mg/kg/day),
CLR (7.15 mg/kg/day),
PPI (138 mg/kg/day),
G6: 100 mg/kg/day,
G7: 200 mg/kg/day, and
G8: 500 mg/kg/day (1) Gastric Rapid Urease Test (CLO Test)

The rapid urease test is based on the principle according to which, when H. pylori is present in gastric mucosa, the bacteria produce ammonia by secreting urease while proliferating in a medium containing a test reagent and a pH indicator, and a change in the color of the pH indicator is examined.

<Calculation of Therapeutic Rate>

The gastric mucosal tissue extracted on the day of the autopsy was collected aseptically and tested using a Campylobacter-like organism (CLO) test reagent. The tissue was incubated in an incubator at 37° C. for 2 hours, the resulting color changed from yellow to red, the sample was judged as positive. The number of mice determined to be positive was calculated as a percentage, and the therapeutic rate for H. pylori eradication by sample treatment was obtained using the following equation, and the results are shown in Table 2 below.

Therapeutic rate (%)=(number of samples−number of positive samples)/number of samples×100

TABLE 2

| Group | Infection | Sample | Number of mice | Therapeutic % |
|---|---|---|---|---|
| G1 | PBS | Corn oil | 10 | 100% |
| G2 | PBS | 0.5% CMC | 9 | 100% |
| G3 | H. pylori | Corn oil | 10 | 30% |
| G4 | H. pylori | 0.5% CMC | 10 | 40% |
| G5 | H. pylori | MTN + CLR + PPI | 10 | 70% |
| G6 | H. pylori | 100 mg/kg | 10 | 60% |
| G7 | H. pylori | 200 mg/kg | 9 | 56% |
| G8 | H. pylori | 500 mg/kg | 10 | 80% |

As shown in Table 2 above, the therapeutic rate of the G5 antibiotic group was 70%, which was higher than that of the G4 group, but there was no statistically significant difference between the two groups. The therapeutic rates of G6 and G7 were 60% and 56%, respectively, which were higher than that of G3, but there was no statistically significant difference between the groups. It could be confirmed that the therapeutic rate of G8 was 80%, which was statistically significantly higher than that of G3. (2) CLO score

After the CLO test, the CLO score was measured based on the following criteria: 0=there is no change in the color of the medium after the test; 1=the medium shows a little red color; 2=the medium shows light purple; 3=the medium shows purple. The mean and standard deviation of each group were calculated, and the difference between the values of the groups was compared, and the results are shown in Table 3 below.

TABLE 3

| Group | Infection | Sample | Number of mice | CLO score |
|---|---|---|---|---|
| G1 | PBS | Corn oil | 10 | 0.00 |
| G2 | PBS | 0.5% CMC | 9 | 0.00 |
| G3 | H. pylori | Corn oil | 10 | 2.10 |
| G4 | H. pylori | 0.5% CMC | 10 | 1.70 |
| G5 | H. pylori | MTN + CLR + PPI | 10 | 0.9 |
| G6 | H. pylori | 100 mg/kg | 10 | 1.2 |
| G7 | H. pylori | 200 mg/kg | 9 | 1.11 |
| G8 | H. pylori | 500 mg/kg | 10 | 0.5 |

As shown in Table 3 above, the CLO score of the G5 antibiotic group decreased by 47.1% compared to that of G4, but there was no statistically significant difference between the two groups. The CLO scores of G6 and G7 decreased by 42.9% and 47.1%, respectively, compared to that of G3, but there was no statistically significant difference between the groups. The CLO score of G8 decreased by 76.2% compared to that of G3, and thus showed a statistically significant difference. (3) Quantitative polymerase chain (qPCR) test for gastric mucosal Helicobacter pylori

After completion of the test, RNA was collected from the aseptically collected gastric mucosal tissue, and then synthesized into DNA to perform a PCR test. The marker gene used in this experiment was 16S rRNA, which is specifically found only in H. pylori and is not found in humans or mice. 18SrRNA was used as a loading control gene of the used DNA sample. The Ct value for each gene was obtained by reacting the fluorescent intercalator SYBR Green with the corresponding primer and DNA, and then calculated by relative quantification, and the results are shown in Table 4 below.

TABLE 4

| | | | Number of | Gene expression |
|---|---|---|---|---|
| Group | Infection | Sample | mice | level (Ct) |
| G1 | PBS | Corn oil | 10 | 3.77 |
| G2 | PBS | 0.5% CMC | 9 | 4.96 |
| G3 | H. pylori | Corn oil | 10 | 13.80 |
| G4 | H. pylori | 0.5% CMC | 10 | 13.92 |
| G5 | H. pylori | MTN + CLR + PPI | 10 | 10.05 |
| G6 | H. pylori | 100 mg/kg | 10 | 11.99 |
| G7 | H. pylori | 200 mg/kg | 9 | 9.40 |
| G8 | H. pylori | 500 mg/kg | 10 | 5.71 |

As shown in Table 4 above, the Ct value of the G5 antibiotic group decreased by 27.8% compared to that of G4, but there was no statistically significant difference between the two groups. The Ct values of G6 and G7 decreased by 13.1% and 31.9%, respectively, compared to that of G3, but there was no statistically significant difference between the groups. The Ct value of G8 decreased by 58.6% compared to that of G3, and thus showed a statistically significant difference. Experimental Example 4: Human test for Effect of Beta-caryophyllene on Improvement of Helicobacter pylori-Infected People

In order to verify the anti-Helicobacter pylori effect of beta-caryophyllene, beta-caryophyllene was administered to 16 semi-healthy people (Helicobacter pylori-infected people) complaining of gastrointestinal disorders, at a dose of 126 mg/day once a day for 8 weeks before meals, and changes in digestive system symptoms (nausea, epigastric pain, heartburn, acid regurgitation, and indigestion) were evaluated, and the results are shown in Table 5.

TABLE 5

| | Before | After | Improvement |
|---|---|---|---|
| Symptoms | intake | intake | rate (%) |
| Nausea | 0.19 | 0.06 | 68.4 |
| Epigastric pain | 1.31 | 0.44 | 66.4 |
| Heartburn | 0.88 | 0.38 | 56.8 |
| Acid regurgitation | 0.25 | 0.06 | 76 |
| Indigestion | 0.56 | 0.13 | 76.8 |

As shown in Table 5, it can be seen that beta-caryophyllene of the present invention improves Helicobacter pylori-induced gastrointestinal disorders by 56% or more. Although the present invention has been described above with reference to the limited embodiments and drawings, it should be understood that the present invention is not limited to these embodiments, and various modifications and variations may be made by those of ordinary skill in the art to which the present invention pertains, without departing from the technical spirit of the present invention and equivalents to the appended claims.

1. An anti-Helicobacter food composition containing beta-caryophyllene having a purity of 90% or higher, obtained by distilling clove oil at 250 to 270° C., in an amount corresponding to a daily adult intake of 100 to 2,500 mg.

2. The anti-Helicobacter food composition according to claim 1, wherein the anti-Helicobacter food composition is mixed or diluted with a carrier or encapsulated in a capsule form.

3. The anti-Helicobacter food composition according to claim 2, wherein the carrier is selected from the group consisting of lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil.

4. A lactic acid bacteria-fermented food containing beta-caryophyllene as an active ingredient in yogurt or lactic acid-bacteria-fermented milk, wherein the beta-caryophyllene is obtained by distilling clove oil at 250 to 270° C., has a purity of 90% or higher, and is contained in an amount corresponding to a daily adult intake of 100 to 2,500 mg.
